SEQUENCE LISTING (2 sequences)

SEQ ID NO: 1 - protein, 391 aa, Artificial Sequence
  CK2 Peptide
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 2 - protein, 17 aa, Artificial Sequence
  OGT Substrate Peptide
KKKYPGGSTPVSSANMM

(19) United States
(12) Patent Application Publication
(10) Pub. No.: US 2021/0109103 A1
(43) Pub. Date: Apr. 15, 2021

(54) METHODS AND KITS FOR DETECTING O-GlcNAc SITES USING B3GALNT2 AND OGT

(71) Applicant: BIO-TECHNE CORPORATION, Minneapolis, MN (US)

(72) Inventors: Zhengliang L. Wu, Edina, MN (US); Tim Tatge, Richfield, MN (US); Yonglong Zou, Shoreview, MN (US); Alex Grill, New Brighton, MN (US)

(21) Appl. No.: 17/047,890

(22) PCT Filed: Apr. 17, 2019
(86) PCT No.: PCT/US2019/027978

§ 371 Date: Oct. 15, 2020

Related U.S. Application Data
(60) Provisional application No. 62/658,913, filed on Apr. 17, 2018.

(51) Int. Cl. G01N 33/58

(52) U.S. Cl. CPC G01N 33/581; G01N 2400/00

(57) ABSTRACT

In an in vitro method of determining the modification degree of O-GlcNAc in a biological sample, a first sample is treated with B3GALNT2 to incorporate a GalNAz into closed O-GlcNAc sites in the sample, labeled using click chemistry to form labeled closed O-GlcNAc sites, and the labeled closed O-GlcNAc sites in the first sample are detected (corresponding to the number of closed O-GlcNAc sites in the biological sample). A second, duplicate sample is treated with OGT to O-GlcNAcylate open O-GlcNAc sites in the sample to convert the open O-GlcNAc sites to closed O-GlcNAc sites, and treated with B3GALNT2 to incorporate GalNAz into the closed O-GlcNAc sites. The second sample is labeled using click chemistry to form labeled closed O-GlcNAc sites (corresponding to the total number of O-GlcNAc sites in the biological sample). The number of closed O-GlcNAc sites and total O-GlcNAc sites in the biological sample are compared to determine the modification degree of O-GlcNAc.

### RELATED MATTERS

This application claims priority to U.S. Provisional Application No. 62/658,913, filed Apr. 17, 2018, the entire contents of which are incorporated herein by reference.

### TECHNICAL FIELD

This disclosure relates to O-GlcNAc, and more specifically, detecting O-GlcNAc sites in a biological sample using B3GALNT2 and O-GlcNAc transferase (OGT).

### BACKGROUND

O-GlcNAc post-translational modification (O-GlcNAcylation) refers to a single β-N-acetylglucosamine residue attached to serine/threonine residues (O-GlcNAc sites) on nuclear and cytosolic proteins. O-GlcNAcylation is a reversible serine/threonine glycosylation for regulating protein activity and availability inside of cells. O-GlcNAc is involved in many cellular processes, including transcription, translation, cell signaling and cell cycle regulation, and is therefore critical for cell growth, migration and differentiation. O-GlcNAcylated serine/threonine residues and unmodified serine/threonine residues that can be O-GlcNAcylated are inversely related, and the balance between them could be finely tuned in a biological system to achieve an optimal level for the best performance of the target proteins. For example, in casein kinase 2 (CK2), O-GlcNAcylation at Ser347 regulates its kinase substrate specificity while making the protein permissive to proteasomal degradation.

Various methods for O-GlcNAc detection have been explored. For example, radioisotope methods for O-GlcNAc detection include incorporation of [³H]-Gal using galactosyltransferase and incorporation of [³⁵S]-SO₃ using carbohydrate sulfotransferases, CHST2 and CHST4. Known chemical methods for O-GlcNAc labeling include incorporation of modified Gal or GalNAc or GlcNAc using recombinant galactosyltransferases or through metabolic pathways. Additionally, O-GlcNAc antibodies and O-GlcNAc binding proteins have been developed for identification of O-GlcNAc modified proteins. However, these methods lack specificity for O-GlcNAc and can be inconvenient to perform. In addition, there is no known method for detecting unmodified (open) O-GlcNAc sites.

### SUMMARY

In general, this disclosure relates to detecting and imaging O-GlcNAc and O-GlcNAc sites in a biological sample, such as purified proteins, cells, cellular extract, or tissue. As used herein, “open O-GlcNAc sites” refers to those sites that can be occupied by O-GlcNAc, meaning they can be O-GlcNAcylated. “Closed O-GlcNAc sites” or “O-GlcNAC” refers to those sites that are occupied by O-GlcNAc, meaning they have already been O-GlcNAcylated.

This disclosure is advantageous, because it provides methods of detecting both closed and open O-GlcNAc sites in a biological sample by using B3GALNT2 and OGT. Being able to detect and differentiate between closed and open O-GlcNAc sites allows for determination of the degree of post-translational modification (“modification degree”) of O-GlcNAc in a biological sample. As used herein, “degree of post-translational modification” or “modification degree” refers to the percentage of closed O-GlcNAc sites as compared to the total O-GlcNAc sites in a sample. The modification degree of O-GlcNAc in a biological sample can help determine if there is abnormally low or high O-GlcNAcylation in a biological sample, which can assist in diagnosing and treatment of diseases, such as diabetes and cancers.

In one embodiment, an in vitro method of detecting closed O-GlcNAc sites in a biological sample includes providing a biological sample and treating the sample with B3GALNT2 to incorporate a GalNAz into the closed O-GlcNAc sites in the sample. The GalNAz includes a click chemistry moiety. The method further includes adding a label to the sample that includes a click chemistry moiety that reacts to the click chemistry moiety of the GalNAz such that the label attaches to the GalNAz to form labeled closed O-GlcNAc sites. The method further includes detecting the labeled closed O-GlcNAc sites.

In another embodiment, an in vitro method of detecting open O-GlcNAc sites in a biological sample includes providing a biological sample and treating the sample with OGT to incorporate GlcNAz into the open O-GlcNAc sites in the sample. The GlcNAz includes a click chemistry moiety. The method further includes adding a label to the sample that includes a click chemistry moiety that reacts to the click chemistry moiety of the GlcNAz such that the label attaches to the GlcNAz to form labeled open O-GlcNAc sites. The method further includes detecting the labeled open O-GlcNAc sites.

In another embodiment, an in vitro method of detecting total O-GlcNAc sites in a biological sample includes providing a biological sample, treating the sample with OGT to O-GlcNAcylate open O-GlcNAc sites in the sample to convert the open O-GlcNAc sites to closed O-GlcNAc sites, and treating the sample with B3GALNT2 to incorporate GalNAz into the closed O-GlcNAc sites in the sample. The GalNAz includes a click chemistry moiety. The method further includes adding a label to the sample that includes a click chemistry moiety that reacts to the click chemistry moiety of the GalNAz such that the label attaches to the GalNAz to form labeled closed O-GlcNAc sites. The method further includes detecting the labeled closed O-GlcNAc sites. The labeled O-GlcNAc sites correspond to the total O-GlcNAc sites in the biological sample.

In another embodiment, an in vitro method of determining the degree of post-translational modification of O-GlcNAc in a biological sample includes obtaining a first sample and a second, duplicate sample from a biological sample and treating the first sample with B3GALNT2 to incorporate a GalNAz into closed O-GlcNAc sites in the first sample. The GalNAz includes a click chemistry moiety. The method further includes adding a label to the first sample that includes a click chemistry moiety that reacts to the click chemistry moiety of the GalNAz such that the label attaches to the GalNAz to form labeled closed O-GlcNAc sites. The method further includes detecting the labeled closed O-GlcNAc sites in the first sample. The labeled closed O-GlcNAc sites in the first sample correspond to the number of closed O-GlcNAc sites in the biological sample. The method further includes treating the second sample to O-GlcNAcylate open O-GlcNAc sites in the second sample to convert the open O-GlcNAc sites to closed O-GlcNAc sites, and treating the second sample with B3GALNT2 to incorporate a GalNAz into the closed O-GlcNAc sites in the second sample. The GalNAz includes a click chemistry moiety. The method further includes adding a label to the second sample that includes a click chemistry moiety that reacts to the click chemistry moiety of the GalNAz such that the label attaches to the GalNAz to form labeled closed O-GlcNAc sites, and detecting the labeled closed O-GlcNAc sites in the sample. The labeled closed O-GlcNAc sites in the second sample correspond to the total O-GlcNAc sites in the biological sample. The method further includes comparing the number of closed O-GlcNAc sites in the biological sample to the total number of O-GlcNAc sites in the biological sample to determine a percentage of closed O-GlcNAc sites in the biological sample. The percentage corresponds to the modification degree of O-GlcNAc in the biological sample.

In another embodiment, an in vitro method of diagnosing diabetes includes obtaining a first sample and a second, duplicate sample from a biological sample from a patient, and treating the first sample with B3GALNT2 to incorporate a GalNAz into closed O-GlcNAc sites in the first sample. The GalNAz includes a click chemistry moiety. The method further includes adding a label to the first sample that includes a click chemistry moiety that reacts to the click chemistry moiety of the GalNAz such that the label attaches to the GalNAz to form labeled closed O-GlcNAc sites. The method further includes detecting the labeled closed O-GlcNAc sites in the first sample. The labeled closed O-GlcNAc sites in the first sample correspond to the number of closed O-GlcNAc sites in the biological sample. The method further includes treating the second sample to O-GlcNAcylate open O-GlcNAc sites in the second sample to convert the open O-GlcNAc sites to closed O-GlcNAc sites, and treating the second sample with B3GALNT2 to incorporate a GalNAz into the closed O-GlcNAc sites in the second sample. The GalNAz includes a click chemistry moiety. The method further includes adding a label to the second sample that includes a click chemistry moiety that reacts to the click chemistry moiety of the GalNAz such that the label attaches to the GalNAz to form labeled closed O-GlcNAc sites, and detecting the labeled closed O-GlcNAc sites in the sample. The labeled closed O-GlcNAc sites in the second sample correspond to the total O-GlcNAc sites in the biological sample. The method further includes comparing the number of closed O-GlcNAc sites in the biological sample to the total number of O-GlcNAc sites in the biological sample to determine a percentage of closed O-GlcNAc sites in the biological sample. The percentage corresponds to the modification degree of O-GlcNAc in the biological sample. The method further includes diagnosing the patient with diabetes if the modification degree of O-GlcNAc in the biological sample meets a threshold modification degree of O-GlcNAc.

In another embodiment, an in vitro method of diagnosing cancer includes obtaining a first sample and a second, duplicate sample from a biological sample from a patient, and treating the first sample with B3GALNT2 to incorporate a GalNAz into closed O-GlcNAc sites in the first sample. The GalNAz includes a click chemistry moiety. The method further includes adding a label to the first sample that includes a click chemistry moiety that reacts to the click chemistry moiety of the GalNAz such that the label attaches to the GalNAz to form labeled closed O-GlcNAc sites. The method further includes detecting the labeled closed O-GlcNAc sites in the first sample. The labeled closed O-GlcNAc sites in the first sample correspond to the number of closed O-GlcNAc sites in the biological sample. The method further includes treating the second sample to O-GlcNAcylate open O-GlcNAc sites in the second sample to convert the open O-GlcNAc sites to closed O-GlcNAc sites, and treating the second sample with B3GALNT2 to incorporate a GalNAz into the closed O-GlcNAc sites in the second sample. The GalNAz includes a click chemistry moiety. The method further includes adding a label to the second sample that includes a click chemistry moiety that reacts to the click chemistry moiety of the GalNAz such that the label attaches to the GalNAz to form labeled closed O-GlcNAc sites, and detecting the labeled closed O-GlcNAc sites in the sample. The labeled closed O-GlcNAc sites in the second sample correspond to the total O-GlcNAc sites in the biological sample. The method further includes comparing the number of closed O-GlcNAc sites in the biological sample to the total number of O-GlcNAc sites in the biological sample to determine a percentage of closed O-GlcNAc sites in the biological sample. The percentage corresponds to the modification degree of O-GlcNAc in the biological sample. The method further includes diagnosing the patient with cancer if the modification degree of O-GlcNAc in the biological sample meets a threshold modification degree of O-GlcNAc.

In another embodiment, a kit for in vitro detection of closed O-GlcNAc sites in a biological sample includes B3GALNT2, UDP-GalNAz with a click chemistry moiety, a label including a click chemistry moiety that reacts to the click chemistry moiety of the GalNAz, and click chemistry reagents.

In another embodiment, a kit for in vitro detection of open O-GlcNAc sites in a biological sample includes OGT, UDP-GlcNAz with a click chemistry moiety, a label including a click chemistry moiety that reacts to the click chemistry moiety of the GlcNAz, and click chemistry reagents.

In another embodiment, a kit for in vitro detection of total O-GlcNAc sites in a biological sample includes OGT, B3GALNT2, UDP-GlcNAc, UDP-GalNAz with a click chemistry moiety, a label including a click chemistry moiety that reacts to the click chemistry moiety of the GalNAz, and click chemistry reagents.

### BRIEF DESCRIPTION OF DRAWINGS

FIG. 1 is a flow diagram of an overview of methods of detecting closed, open, and total O-GlcNAc sites in a biological sample according to exemplary embodiments.

FIG. 2 is a flow diagram of a method of detecting closed O-GlcNAc sites in a biological sample according to an exemplary embodiment.

FIG. 3 is a flow diagram of a method of detecting open O-GlcNAc sites in a biological sample according to an exemplary embodiment.

FIG. 4 is a flow diagram of a method of detecting total O-GlcNAc sites in a biological sample according to an exemplary embodiment.

FIG. 5 is a flow diagram of a method of determining the modification degree of O-GlcNAc in a biological sample according to an exemplary embodiment.

FIG. 6 is the sequence listing of a CK2 peptide, showing the location of the O-GlcNAc site at Ser347, along with the corresponding sequence of an OGT peptide substrate.

FIGS. 7A-7C are mass spectrum graphs of a CK2 peptide (FIG. 7A), an O-GlcNAcylated CK2 peptide (FIG. 7B), and a CK2 peptide treated with both OGT and B3GALNT2 in the presence of UDP-GlcNAc and UDP-GalNAc (FIG. 7C).

FIG. 8 is a bar graph of the activity of B3GALNT2 and OGT on regular nucleotide sugars compared to azido nucleotide sugars.

FIGS. 9A-9B are Western blots of recombinant CK2 that was probed for O-GlcNAc sites using OGT and B3GALNT2.

FIGS. 10A-10B are Western blots of cellular extract that was probed for O-GlcNAc sites using OGT and B3GALNT2.

FIG. 11 is an image of CHO-K1 cells imaged for closed O-GlcNAc sites with B3GALNT2.

FIG. 12 is an image of CHO-K1 cells imaged for open O-GlcNAc sites with OGT.

FIG. 13 is an image of CHO-K1 cells imaged for total O-GlcNAc sites with B3GALNT2 after treatment with OGT in the presence of UDP-GlcNAc.

FIG. 14 is an image of CHO-K1 cells imaged for closed O-GlcNAc sites with B3GALNT2 after treatment with OGA.

### DETAILED DESCRIPTION

The following detailed description is exemplary in nature and is not intended to limit the scope, applicability, or configuration of the disclosure in any way. Unless defined otherwise, all technical and scientific terms used herein have the same meaning as those commonly understood by one of ordinary skill in the art to which this invention belongs. Although methods and materials similar or equivalent to those described herein may be used in the invention or testing, suitable methods and materials are described herein. The materials, methods and examples are illustrative only, and are not intended to be limiting. Those skilled in the art will recognize that many of the noted examples have a variety of suitable alternatives. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

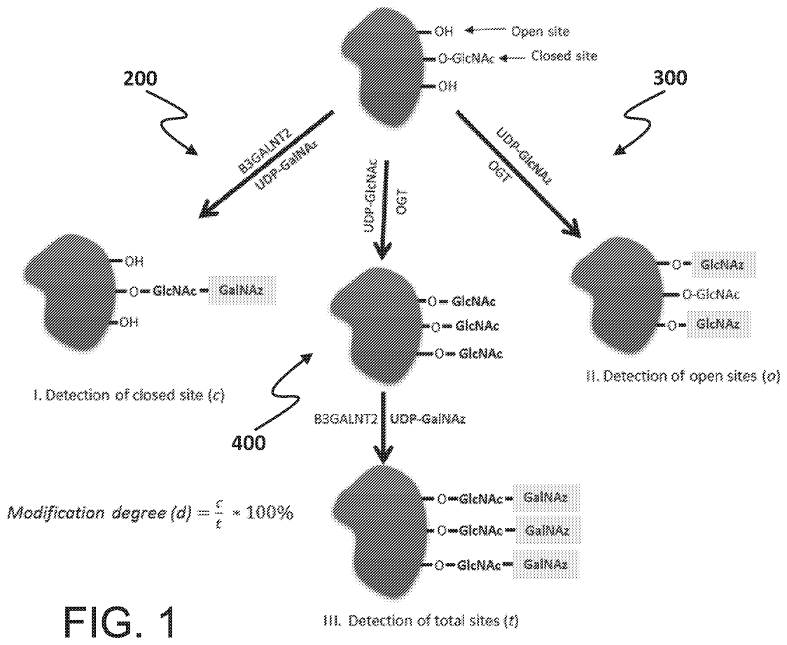

FIG. 1 is a flow diagram of an overview of in vitro methods 200, 300, and 400 of detecting closed, open, and total O-GlcNAc sites in a biological sample according to exemplary embodiments. Method 200 detects closed O-GlcNAc sites. Method 300 detects open O-GlcNAc sites. Method 400 detects total O-GlcNAc sites.

O-GlcNAcylation is a reversible serine/threonine glycosylation for regulating protein activity and availability inside of cells. Again, O-GlcNAcylated sites inside of cells are referred to as closed sites and unoccupied O-GlcNAc sites are referred to as open sites. O-GlcNAcylation regulates energy metabolism. For example, dysregulation of O-GlcNAcylation in cells is related to insulin resistance in diabetes or etiology of cancer. Thus, regulation between open and closed sites is believed to be dynamic and indicative of various statuses of cells in the biological sample.

Methods 200, 300, and 400 provide a new approach for assessing open and closed O-GlcNAc sites in biological samples using two detection enzymes. The two detection enzymes are glycosyltransferases, namely OGT (O-GlcNAc transferase) and B3GALNT2 (0-1,3-N-acetylgalactosaminyltransferase). Applicant has discovered that these two detection enzymes allow for more highly specific methods of detecting and assessing open and closed O-GlcNAc sites. Therefore, the methods 200, 300, and 400 allow for highly specific assessment of closed sites, open sites and total sites, which in turn can indicate cellular status.

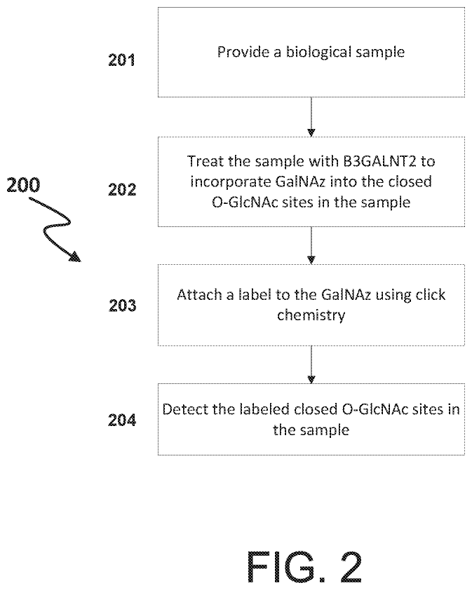

FIG. 2 is a flow diagram of in vitro method 200 of detecting closed O-GlcNAc sites in a biological sample. Method 200 includes steps of providing a biological sample (201), treating the biological sample with B3GALNT2 to incorporate GalNAz into closed O-GlcNAc sites in the biological sample (202), attaching a clickable label to the GalNAz using click chemistry (203) and detecting the clickable label (204). If the clickable label is detected, there are closed O-GlcNAc sites in the sample. If no clickable label is detected, there are no closed O-GlcNAc sites in the sample.

Step 201 includes providing a biological sample. The biological sample can comprise any sample that includes cells and wherein it is desired to study O-GlcNAcylation inside cells of the sample. For example, the biological sample includes a purified protein, a whole cell, a cellular extract or tissue. In certain cases, the biological sample includes a purified nuclear protein. In other cases, the biological sample includes a purified cytosolic protein. The biological sample can be isolated and prepared using known methods.

Step 202 includes treating the biological sample with B3GALNT2 to incorporate GalNAz into closed O-GlcNAc sites. B3GALNT2 is a β-1,3-N-acetylgalactosaminyltransferase that synthesizes a unique carbohydrate structure, GalNAc-β1,3-GlcNAc, on N- and O-glycans and the phosphorylated O-mannosyl trisaccharide (GalNAc-β-3-GlcNAc-β-4-(phosphate-6-)Man). In some cases, the B3GALNT2 is recombinant B3GALNT2. In other cases, the B3GALNT2 is recombinant human B3GALNT2. Recombinant human B3GALNT2 in some cases can be obtained from R&D Systems® (Minneapolis, Minn.).

Applicant has discovered that B3GALNT2 recognizes closed O-GlcNAc sites. Therefore, by treating the biological sample with B3GALNT2 to incorporate GalNAz into closed O-GlcNAc sites of cells in the biological sample, one can then use click chemistry to detect closed O-GlcNAc sites. GalNAz is a clickable carbohydrate. GalNAz includes an azido group, which is a click chemistry moiety that can be used in a click chemistry reaction. In certain cases, the source of the GalNAz is UDP-GalNAz.

Step 203 includes adding a clickable label to the biological sample. The clickable label includes a click chemistry moiety that reacts to the click chemistry moiety of the GalNAz such that the clickable label attaches to the GalNAz to form labeled closed O-GlcNAc sites. Step 204 includes detecting labeled closed O-GlcNAc sites in the biological sample. The clickable label can be any clickable label known in the art that can be detected by a detection method, such as an imaging method, a Western blotting method, or an enzyme-linked immunosorbent assay (ELISA) method. For example, in some embodiments, the clickable label can be a reporter molecule, such as a fluorescent label, a colorimetric label, an enzyme label, a biotin conjugate linked to a fluorescent label, a biotin conjugate linked to a colorimetric label, or a biotin conjugate linked to an enzyme label (such as the reporter enzyme horseradish peroxidase). In cases where the detection method is an imaging method, an imaging device known in the art can be used to detect the clickable label. In some cases, the label is a fluorescent label and the imaging device is a fluorescent imaging device, such as a fluorescent camera. In other cases, the label is a colorimetric label and the imaging device is a colorimetric camera.

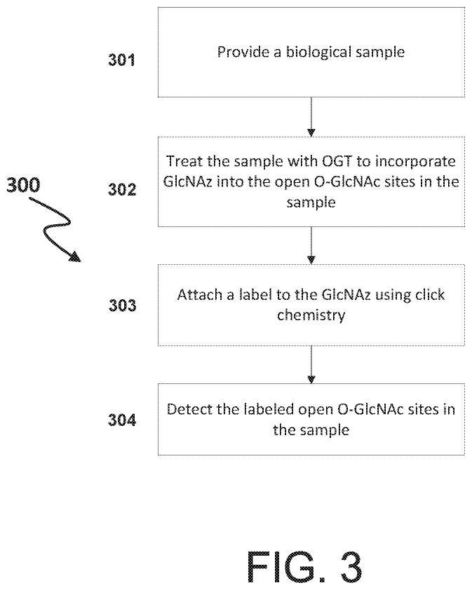

FIG. 3 is a flow diagram of in vitro method 300 of detecting open O-GlcNAc sites in a biological sample. The method 300 includes providing a biological sample (301), treating the biological sample with OGT to incorporate GlcNAz into open O-GlcNAc sites (302), attaching a clickable label to the GlcNAz using click chemistry (303) and detecting the clickable label (304). If clickable label is detected, there are open O-GlcNAc sites in the sample. If no clickable label is detected, there are no open O-GlcNAc sites.

Step 301 includes providing a biological sample. The biological sample can be any biological sample as described herein. Step 302 includes treating the biological sample with OGT (O-GlcNAc transferase) to incorporate GlcNAz into open O-GlcNAc sites in the biological sample. In some cases, the OGT is recombinant OGT. In other cases, the OGT is recombinant human OGT. Recombinant human OGT in some cases can be obtained from R&D Systems® (Minneapolis, Minn.).

Applicant has discovered that OGT is highly specific for open O-GlcNAc sites. Therefore, by treating the biological sample with OGT to incorporate GlcNAz into open O-GlcNAc sites, one can then use click chemistry to detect open O-GlcNAc sites. GlcNAz is a clickable carbohydrate. GlcNAz includes an azido group, which is a click chemistry moiety that can be used in a click chemistry reaction. In certain cases, the source of the GlcNAz is UDP-GlcNAz.

Step 303 includes adding a clickable label to the sample. The clickable label includes a click chemistry moiety that reacts to the click chemistry moiety of the GlcNAz such that the clickable label attaches to the GlcNAz to form labeled open O-GlcNAc sites. Step 304 includes detecting labeled open O-GlcNAc sites in the biological sample. Again, the clickable label can be any clickable label known in the art that can be detected by a detection method, such as an imaging method, a Western blotting method, or an ELISA method.

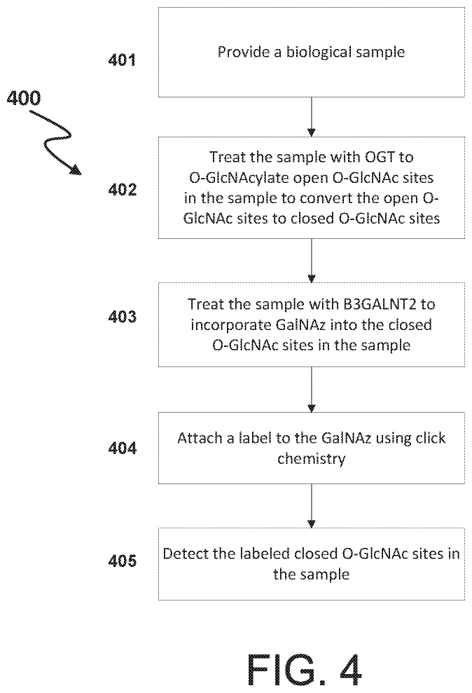

FIG. 4 is a flow diagram of an in vitro method 400 of detecting total O-GlcNAc sites in a biological sample. The method 400 includes providing a biological sample (401), treating the biological sample with OGT to O-GlcNAcylate open O-GlcNAc sites to convert open O-GlcNAc sites to closed O-GlcNAc sites in the biological sample (402), treating the biological sample with B3GALNT2 to incorporate GalNAz into O-GlcNAc sites in the biological sample (403), attaching a clickable label to the GalNAz using click chemistry (404) and detecting labeled closed O-GlcNAc sites in the biological sample (405).

Step 401 includes providing a biological sample, which can be any biological sample as described herein. Step 402 includes treating the biological sample with OGT to O-GlcNAcylate open O-GlcNAc sites (and therefore converting open sites to closed sites). Step 402 should result in the conversion of any open site in the biological sample to a closed site, such that all or substantially all O-GlcNAc sites in the sample are closed sites. The OGT can be according to any embodiment described for OGT herein.

Step 403 includes treating the biological sample with B3GALNT2 to incorporate GalNAz into the closed O-GlcNAc sites in the sample. The B3GALNT2 and GalNAz can be according to any embodiment described for B3GALNT2 and GalNAz herein. Step 404 includes adding a clickable label to the sample. The clickable label includes a click chemistry moiety that reacts to the click chemistry moiety of the GalNAz such that the clickable label attaches to the GalNAz to form labeled closed O-GlcNAc sites. Step 405 includes detecting labeled closed O-GlcNAc sites in the biological sample. Again, the clickable label can be any clickable label known in the art that can be detected by a detection method, such as an imaging method or a Western blotting method. Because step 402 converts any open site in the biological sample to a closed site, such that all or substantially all O-GlcNAc sites in the sample are closed sites, the labeled closed O-GlcNAc sites detected in step 405 correspond to the total O-GlcNAc sites in the biological sample.

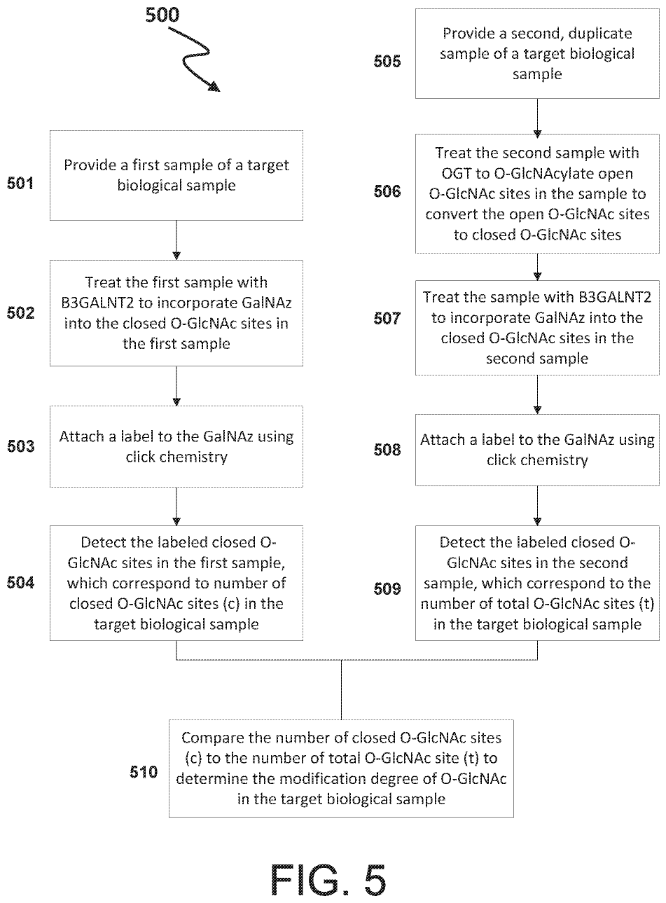

FIG. 5 is a flow diagram of a method of determining the modification degree of O-GlcNAc in a target biological sample. The method 500 includes providing a first biological sample of a target biological sample (501), treating the first biological sample with B3GALNT2 to incorporate GalNAz into closed O-GlcNAc sites in the first sample (502), attaching a clickable label to the GalNAz using click chemistry (503), and detecting the clickable label to obtain a first value (c) corresponding to number of closed O-GlcNAc sites in the target biological sample (504). The method 500 further includes providing a second, duplicate biological sample of the target biological sample (505), treating the second, duplicate biological sample with OGT to O-GlcNAcylate open O-GlcNAc sites to convert open O-GlcNAc sites to closed O-GlcNAc sites (506), treating the second, duplicate sample with B3GALNT2 to incorporate GalNAz into the closed O-GlcNAc sites in the second sample (507), attaching a clickable label to the GalNAz using click chemistry (508), and detecting the clickable label to obtain a second value (t) corresponding to number of total O-GlcNAc sites in the target biological sample (509). The method 500 further includes comparing the first value (c) to the second value (t) to determine a modification degree (d) of O-GlcNAc in the target biological sample (510). The first biological sample and the second, duplicate biological sample are both obtained from the target biological sample. The first sample and the second, duplicate sample are prepared such that they have substantially the same amount or concentration of biological material such that the number of total O-GlcNAc sites in the first sample and the second sample is substantially the same.

In certain embodiments, the step of comparing the first value (c) to the second value (t) to determine the modification degree (d) comprises inputting the first value (c) and the second value (t) into the following formula:

Modification degree (d)=c/t*100%

It is desirable to determine a particular modification degree of the target biological sample to assist in assessing (e.g., predicting, diagnosing, staging) a particular disease of the target biological sample. For example, since O-GlcNAc regulates the energy metabolism and dysregulation of O-GlcNAcylation is related insulin resistance in diabetes, modification degree could be used to determine metabolic status and/or assist in the diagnosis or prognosis of diabetes. Thus, in some cases, the target biological sample is assessed for diabetes. Here, the biological sample can comprise a blood sample, liver tissue, or kidney tissue. Therefore, the method in some embodiments includes steps of obtaining a target biological sample from a patient and diagnosing the patient with diabetes if the modification degree (d) of O-GlcNAc meets a threshold modification degree. The method can further include treating the patient for diabetes.

Additionally, dysregulation of O-GlcNAcylation is also related to the etiology of cancer, so modification degree could be used to determine metabolic status and/or assist in the diagnosis or prognosis of cancer. Thus, in other cases, the target biological sample is assessed for cancer. The target biological sample can therefore comprise material taken from a biopsy sample or tumor sample. In such cases, the method further includes diagnosing a patient with cancer if the modification degree (d) meets a threshold modification degree. The method can further include treating the patient for cancer.

In some embodiments, the modification degree can be indicative of a metabolic status of the target biological sample. In other cases, the modification degree can be indicative of cell growth. In other cases, the modification degree can be indicative of cell migration. In further cases, the modification degree can be indicative of cell differentiation.

### EXAMPLES

Exemplary embodiments of methods 200, 300, 400, and 500 are described in the following Examples.

### Materials

UDP-GlcNAz (advertised as UDP-azido-GlcNAc), UDP-GalNAz (advertised as UDP-azido-GalNAc), recombinant human B3GALNT2, recombinant human O-GlcNAc transferase (OGT), recombinant B. thetaiotaomicron O-GlcNAcase (OGA), biotin alkyne adduct, streptavidin conjugated horseradish peroxidase (strep-HRP), and 4′,6-diamidino-2-phenylindole (DAPI) were obtained from R&D Systems® and/or Bio-Techne®. Streptavidin-Alexa Fluor® 555 and enhanced chemiluminescence (ECL) peroxidase substrate were obtained from Thermo Fisher Scientific®. UDP-GlcNAc was obtained from Sigma-Aldrich®. Benzyl-fl-GlcNAc was obtained from Santa Cruz Biotechnology®. OGT peptide substrate (AS-63726, having sequence KKKYPGGSTPVSSANMM) was obtained from AnaSpec®.

Purification of E. coli Expressed Recombinant CK2

Recombinant human casein kinase II-alpha from amino acid 253 to 391 (rhCK2) was cloned and expressed in E. coli with a C-terminal His tag. A cell lysate of 6 liters of CK2 transfected cells (prepared in a buffer of 25 millimolar (mM) Tris at pH 7.5 and 150 mM NaCl, with 5 parts-per-million (ppm) of protease inhibitor phenylmethylsulfonyl fluoride (PMSF)) was sonicated for 20 minutes with a Fisherbrand™ Model 505 Sonic Dismembrator at 30-40% energy output. The lysate was then centrifuged at 12,000 revolutions per minute (rpm) for 30 minutes. The pellet resulting from centrifugation was collected and resuspended in a buffer of 25 mM Tris at pH 7.5 and 150 mM NaCl, and centrifuged again at 12000 rpm. The resulting pellet was again resuspended and centrifuged two more times. The resulting pellet was subsequently solubilized in 150 milliliters (mL) of 7 M guanidine HCL, 20 mM Tris at pH 7.5, and loaded onto an ÄKTA™ chromatography system with a 20 mL nickel affinity column in order to bind the CK2 in the solution. The bound CK2 was then eluted with 200 mL of a 25 mM and 0.3 M imidazole solution with 6 M urea, and 200 mL of 0.5 M NaCl, 25 mM MES at pH 6.5. Fractions containing CK2 were collected and dialyzed in 2 M urea and 0.5 M NaCl, 25 mM MES at pH 6.5.

### Labeling Reactions

A labeling buffer containing 25 mM Tris at pH 7.5, 10 mM of MnCl₂, and 150 mM NaCl was prepared. In order to O-GlcNAcylate any open O-GlcNAc sites in a sample using OGT, a sample of 30 microliters (4) of cellular extract was mixed with 25 nanomoles (nmol) UDP-GlcNAc and 1 microgram (μg) of OGT, supplemented with 10 μL of labeling buffer, and incubated at 37° C. for 20 minutes. In order to remove any O-GlcNAc residues from a sample using OGA, a sample of 30 μL of cellular extract was mixed with 10 μL of 0.1 M MES at pH 5.5 and 1 μg of OGA, and incubated at 37° C. for 20 minutes.

In order to incorporate GlcNAz into open O-GlcNAc sites in protein sample using OGT, a mixture of 4-10 μg of protein, 5 nmol of UDP-GlcNAz and 1 μg of OGT was supplemented with labeling buffer to obtain a final volume of 50 μL and incubated at 37° C. for 30 minutes. In order to incorporate GalNAz into closed O-GlcNAc sites in a sample using B3GALNT2, a mixture of 4-10 μg of protein, 25 nmol of UDP-GlcNAc, 1 μg of OGT, 5 nmol of UDP-GalNAz and 1 μg of B3GALNT2 was supplemented with labeling buffer to final of 50 μL, and incubated at 37° C. for 30 minutes. After the incorporation of GlcNAz or GalNAz into open or closed O-GlcNAc sites, respectively, a biotin moiety was conjugated to the GlcNAz or GalNAz via a click chemistry reaction to form labeled closed O-GlcNAc sites and labeled open O-GlcNAc sites, respectively. For each reaction, 2 mM ascorbic acid, 0.1 mM CuCl₂ and 0.1 mM biotin alkyne adduct were directly added to the labeling reaction mixture, and the mixture was incubated at room temperature for 30 minutes.

### Western Blotting

Once the click chemistry reaction was complete, the samples were separated on a 12% SDS-PAGE gel or a 4-20% gradient SDS-PAGE gel. The gels were visualized with UV in the presence of trichlorethanol, which reacts with the indole ring of the amino acid tryptophan. Next, the gels were blotted onto nitrocellulose paper under 25 volts for 30 minutes. The blots were then blocked with 10% fat-free milk for 10 minutes, washed thoroughly with TBS buffer (25 mM Tris, pH 7.6, 137 mM NaCl and 0.01% Tween), and subsequently probed with strep-HRP at 30 ng/mL for 30 minutes in TBS buffer. The blots were then washed three times with TBS buffer for a total of 30 minutes. The membrane was visualized with an ECL peroxidase substrate.

### Cell Growth and Fixation

CHO-K1 cells (CCL-61™ from ATCC®) were grown in Iscove's Modified Dulbecco's Media (IMDM) (Thermo Fisher Scientific® Catalog #12440-061), supplemented with 5% fetal bovine serum (Corning® Catalog #35-015-CV), 2 mM L-Glutamine, 100 units/ml penicillin and 0.1 milligram (mg)/mL streptomycin (Sigma-Aldrich® Catalog #G6784). Upon confluence, cells were trypsinized and plated in a 96-well cell culture plate and grown to a desired confluence. The cells were rinsed with sterile phosphate buffered saline (PBS) and fixed in 4% paraformaldehyde for 30 minutes at room temperature followed by washing 5 times with sterile PBS. Upon completion of the washing procedure, the plate was stored in one milliliter sterile PBS at 4° C. until ready for glycan labeling.

### Pretreatment of Cells for Imaging

For glycan imaging, some cells were pretreated with OGA to remove O-GlcNAC residues or pretreated with OGT to O-GlcNAcylate any open O-GlcNAc sites. All of the treatments were applied to cells in a single well of a 96-well plate. For O-GlcNAc removal, for example, cells were treated with 1 μg of OGA in 50 μL of a buffer comprising 50 mM MES, 150 mM NaCl, 0.5% Triton® X-100 at pH 5.5 for 1 hour at 37° C. For O-GlcNAcylation of open O-GlcNAc sites on cells, 50 nmol of UDP-GlcNAc was mixed with 1 μg of OGT in a buffer comprising 25 mM MES, 0.5% (w/v) Triton® X-10, 2.5 mM MgCl₂, 10 mM MnCl₂, 1.25 mM CaCl₂ and 0.75 mg/mL of BSA at pH 7.0 for 1 hour at 37° C. After OGT or OGA treatment, cells were washed thoroughly with phosphate buffered saline (PBS) three times.

Incorporation of Clickable Carbohydrates into O-GlcNAc Sites Using Glycosyltransferases

To incorporate clickable carbohydrates into open or closed O-GlcNAc sites in a cell sample, 25 mM MES, 0.5% (w/v) Triton® X-100, 2.5 mM MgCl₂, 10 mM MnCl₂, 1.25 mM CaCl2 and 0.75 mg/mL of BSA at pH 7.0 was used as a labeling buffer. All of the treatments were applied to cells in a single well of a 96-well plate. For incorporation of GalNAz into closed O-GlcNAc sites, cells were covered with 2 nmol of UDP-GalNAz and 1 μg of B3GALNT2 in 50 μL labeling buffer, and incubated at 37° C. for 30 minutes. For incorporation of GlcNAz into open O-GlcNAc sites, cells were covered with 2 nmol of UDP-GlcNAz and 1 μg of OGT in 50 μl labeling buffer, and incubated at 37° C. for 30 minutes.

### Conjugation of the Clickable Carbohydrate to Biotin and Fluorescent Dye

After the incorporation of the clickable carbohydrate into open or closed O-GlcNAc sites, a biotin moiety was conjugated to the clickable carbohydrate via a click chemistry reaction. For each reaction, 50 μL of 25 mM Tris at pH 7.5, 150 mM NaCl containing 20 nmol of Cu²⁺, 5 nmol of biotin alkyne adduct and 100 nmol of ascorbic acids were combined into a click chemistry mixture and added to each well. The 96-well plate was then incubated at room temperature for 30 minutes. The click chemistry reaction solution was then removed from the 96-well plate and washed thoroughly with PBS. A fluorescent dye mix of 20 μg/mL streptavidin-Alexa Fluor® 555 and 10 μM DAPI in 50 μL of PBS was then applied to the cells for 15 minutes. The streptavidin-Alexa Fluor® 555 bound to the biotin, which resulted in fluorescently labeled open or closed O-GlcNAc sites. The cells were then washed thoroughly with PBS and finally stored in PBS.

### Imaging of the Fluorescently Labeled Cells

All images of the labeled cells were captured on an AXIO Observer microscope (ZEISS) with a ZEISS Axiocam 506 mono camera and Zen 2 Pro software. Images were captured simultaneously through the channels of Alexa Fluor® 555 and/or DAPI. For most images, the exposure time was automatically set and the contrast was adjusted to be a best fit. For imaging through the channel for Alexa Fluor® 555, exposure time was manually set at 4000 ms. For imaging through the channel for DAPI, exposure time was manually set at 140 ms. No adjustment was performed for any image unless indicated.

### Cell Extract Preparation

HEK 293 cells were grown overnight in a 10 cm dish in IMDM supplemented with 5% fetal bovine serum at 37° C. with 5% of CO₂. 1×10⁷ cells were harvested using 5 mL of fresh cold phosphate-buffered saline and centrifuged for 5 min at 450 g to obtain a cell pellet. The cell pellet was gently suspended using 600 μL of isotonic lysis buffer (10 mM TrisHCl at pH 7.5, 2 mM MgCl₂, 3 mM CaCl₂, 0.3 M sucrose, 0.2 mM PMSF and 0.5 mM DTT) and incubated for 15 min on ice. After centrifugation for 5 min at 420 g, the pellet was suspended with 150 μL of isotonic lysis buffer. The cells were then slowly passed through a syringe with a 27 gauge needle 6 times. The lysate was centrifuged for 20 min at 11,000 g and the supernatant (cytoplasmic fraction) was collected. The pellet was then washed with isotonic lysis buffer and extracted using 150 μL extraction buffer (10 mM 4-(2-hydroxyethyl)-1-piperazineethanesulfonic acid at pH 7.9, 1.5 mM MgCl₂, 0.2 mM ethylenediaminetetraacetic acid, 25% Glycerol, 0.42 M NaCl, 0.2 mM PMSF, and 0.5 mM DTT). The lysate was then vortexed and incubated on ice for 30 min. The supernatant (nuclear fraction) was harvested after centrifugation at 21,000 g for 5 minutes. Extracts were stored at −80° C.

### Mass Spectrometry Analysis

Electrospray ionization mass spectrometry (ESI-MS) analysis was performed on an OGT substrate using a Thermo Fisher Scientific® Triple-Stage Quadrupole Mass Spectrometer coupled to an Accela 1250 Pump and Accela Open Autosampler (both from Thermo Fisher Scientific®). 10 μL samples were separated on a Thermo Fisher Scientific® Hypersil GOLD column (50×2.1 mm, 5 μm) using a gradient flow set at 400 mL/min. Mobile phases consisted of A 0.1% formic acid in water and B—0.1% formic acid in acetonitrile. The gradient was 0-1 minute—98% A, 1-20 minutes—98-70% A, 20-22 minutes—70-2% A, 22-23 minutes—2-98% A. Total run time was 30 minutes. The mass analyzer was run in full scan from 400-1200 m/z operating in positive mode. Full MS parameters: DCV=0 V, microscans=1, scan time=1.07 seconds, Q1 PW=0.15 FWHW, spray voltage=4300 V, vaporizer temperature=200° C., sheath gas pressure=35, aux gas pressure=25, capillary temperature=275° C.

### Example 1: Recognition of O-GlcNAc by B3GALNT2

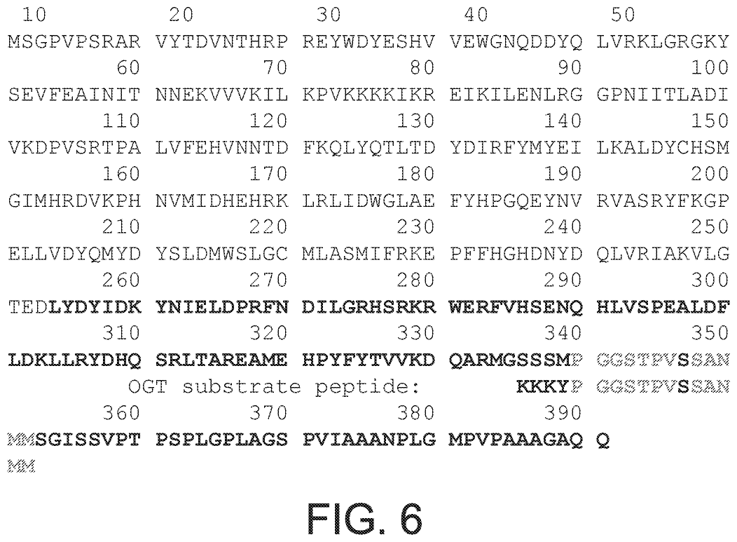

To demonstrate that B3GALNT2 recognizes GlcNAc, a sample peptide that contains the O-GlcNAc site Ser347 of CK2 was first O-GlcNAcylated using OGT, and then treated with B3GALNT2 in the presence of UDP-GalNAc. FIG. 6 is the sequence listing of a CK2 peptide, showing the location of the O-GlcNAc site at Ser347, along with the corresponding sequence of an OGT peptide substrate. After treatment with B3GALNT2, the sample was separated on a C18 column and analyzed using LC-ESI-MS.

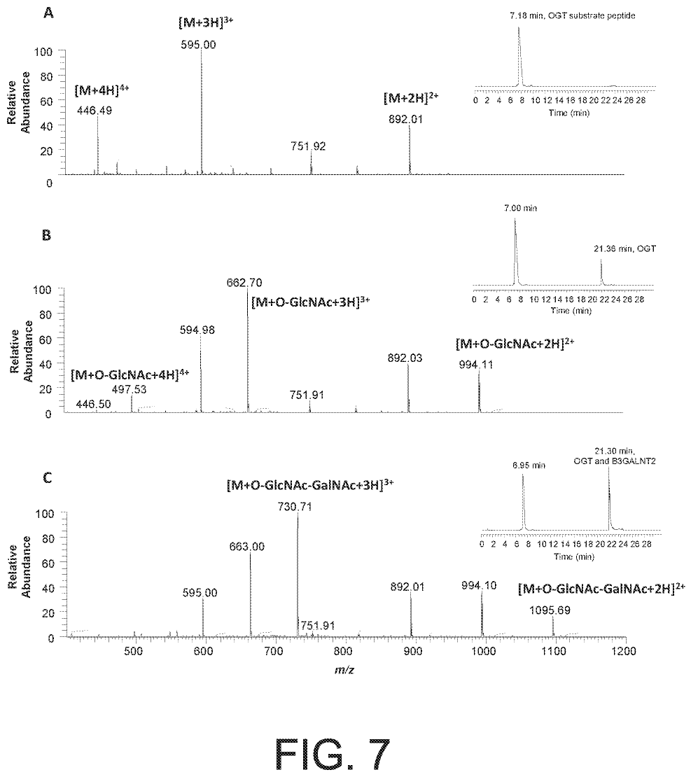

FIGS. 7A-7C are the mass spectrum graphs of a CK2 peptide (FIG. 7A), an O-GlcNAcylated CK2 peptide (FIG. 7B), and a CK2 peptide treated with both OGT and B3GALNT2 in the presence of UDP-GlcNAc and UDP-GalNAc (FIG. 7C). The total ion current chromatogram is shown above each spectrum. The CK2 peptide is eluted around 7.0 minutes and the enzymes are eluted around 21.3 minutes. As can be seen in FIGS. 7A-7C, the mass spectrometry analysis shows that B3GALNT2 adds a GalNAc residue to an O-GlcNAcylated peptide. This confirms that B3GALNT2 recognizes O-GlcNAc.

### Example 2: Comparison of B3GALNT2 and OGT Activity on Regular and Azido Nucleotide Sugar

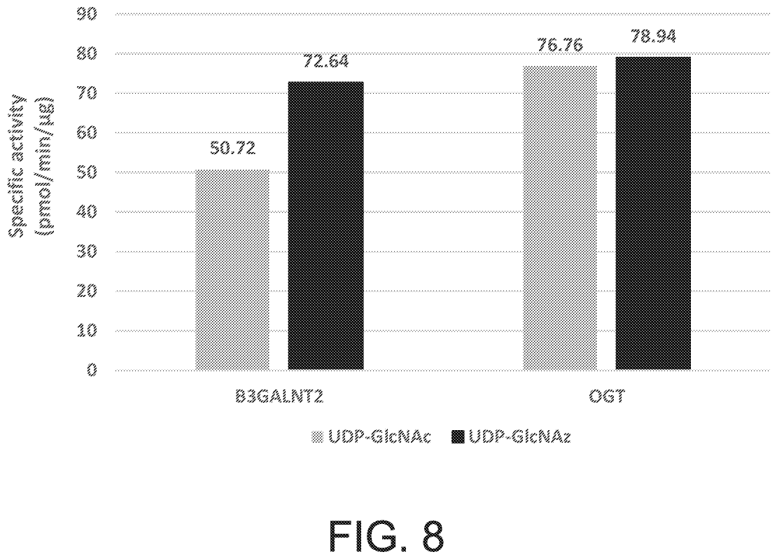

Using a phosphatase coupled universal glycosyltransferase assay, an analysis of the activity of B3GALNT2 and OGT on regular nucleotide sugar as compared to azido nucleotide sugar was performed. For the B3GALNT2 assay, 0.4 mM nucleotide sugar donor (UDP-GalNAc or UDP-GalNAz), 2 mM of acceptor substrate benzyl-β-GlcNAc, and 0.1 μg of coupling phosphatase 1 were combined with 1 μg of B3GALNT2 in 50 μL of assay buffer of 25 mM Tris at pH 7.5, 10 mM MnCl2 and 10 mM CaCl2 at 37° C. for 20 minutes. For the OGT assay, 0.4 mM nucleotide sugar donor (UDP-GlcNAc or UDP-GlcNAz), 0.2 mM of acceptor OGT substrate, and 0.1 μg of coupling phosphatase 1 were combined with 2.5 μg of OGT in 50 μL of assay buffer of 25 mM Tris at pH 7.5, 10 mM MnCl2 and 10 mM CaCl2 at 37° C. for 20 minutes. Both assays were performed according to the Universal Glycosyltransferase Assay Kit from R&D Systems®. FIG. 8 is a bar graph of the activity of B3GALNT2 and OGT on regular nucleotide sugars compared to azido nucleotide sugars. As shown in FIG. 8, the assays showed that B3GALNT2 is more active on UDP-GalNAz than on UDP-GalNAc, and OGT is equally active on UDP-GlcNAz and UDP-GlcNAc.

### Example 3: Western Blot Detection of O-GlcNAc Sites on Recombinant CK2

Applicant discovered that because OGT recognizes open O-GlcNAc sites and can tolerate UDP-GlcNAz, it is possible to use OGT to detect and label open O-GlcNAc sites on proteins. Applicant also discovered that because B3GALNT2 recognizes O-GlcNAc and can tolerate UDP-GalNAz, it is possible to use B3GALNT2 to incorporate GalNAz into O-GlcNAc on target proteins to detect and label closed O-GlcNAc sites on proteins. In order to demonstrate detection and labeling of open O-GlcNAc sites using OGT, E. coli expressed recombinant CK2 was proved with OGT in the presence of UDP-GlcNAz. In order to demonstrate detection and labeling of closed O-GlcNAc sites using OGT and B3GALNT2, E. coli expressed recombinant CK2 was O-GlcNAcylated using recombinant OGT in the presence of UDP-GlcNAc and then probed with B3GALNT2 in the presence of UDP-GalNAz. E. coli expressed recombinant CK2 was also probed with B3GALNT2 in the presence of UDP-GlcNAc and UDP-GalNAz but in the absence of OGT. After the incorporation of GlcNAz or GalNAz into open or closed O-GlcNAc sites, respectively, a biotin moiety (biotin alkyne adduct) was conjugated to the GlcNAz or GalNAz via a click chemistry reaction to form labeled open O-GlcNAc sites and labeled closed O-GlcNAc sites, respectively.

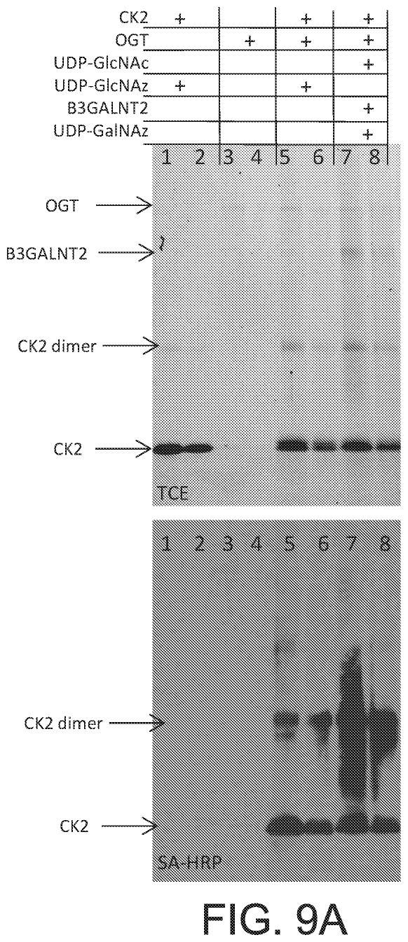

The samples were separated on 4-20% SDS-PAGE gels and visualized with trichloral ethanol (TCE) under UV, as shown in the upper panels of FIGS. 9A-9B. The gels were then blotted to nitrocellulose membranes and detected with strep-HRP, as shown in the lower panels of FIGS. 9A-9B. Lanes 5 and 6 of FIG. 9A and lane 1 of FIG. 9B confirm that OGT detects open O-GlcNAc sites on recombinant CK2 and labels those open sites with UDP-GlcNAz. Lanes 7 and 8 of FIG. 9A and lanes 7 and 8 of FIG. 9B confirm that B3GALNT2 detects closed O-GlcNAc sites on recombinant CK2 and labels those closed sites with UDP-GalNAz.

As shown in lanes 1 and 2 of FIG. 9A, no open O-GlcNAc sites can be detected on recombinant CK2 in the presence of UDP-GlcNAz but without the presence of OGT. As shown in lane 4 of FIG. 9B, no closed O-GlcNAc sites can be detected on recombinant CK2 in the presence of B3GALNT2 but in the absence of UDP-GalNAz. Additionally, lanes 5 and 6 of FIG. 9B show that without O-GlcNAcylation of recombinant CK2 with OGT, even in the presence of UDP-GlcNAc, UDP-GalNAz, and B3GALNT2, no closed O-GlcNAc sites were detected on recombinant CK2. By contrast, when OGT was present, as shown in lanes 7 and 8 of FIG. 9B, closed O-GlcNAc sites were detected and labeled by B3GALNT2. This leads to the conclusion that the recombinant CK2 does not contain any closed O-GlcNAc sites and confirms that B3GALNT2 is highly specific for closed O-GlcNAc sites. These methods of labeling and detecting open and closed O-GlcNAc sites are advantageous, because prior art methods for detecting closed O-GlcNAc sites lack specificity, and until now, there was no known method for detecting open O-GlcNAc sites.

### Example 4: Western Blot Detection of O-GlcNAc Sites on Cellular Extracts

Applicant discovered that because OGT recognizes open O-GlcNAc sites and can tolerate UDP-GlcNAz, it is possible to use OGT to detect and label open O-GlcNAc sites on cellular extracts. Applicant also discovered that because B3GALNT2 recognizes O-GlcNAc and can tolerate UDP-GalNAz, it is possible to use B3GALNT2 to incorporate GalNAz into O-GlcNAc on target proteins to detect and label closed O-GlcNAc sites on cellular extracts. To demonstrate that B3GALNT2 can be used to detect closed O-GlcNAc sites on unknown proteins in cellular extracts, nuclear and cytoplasmic extracts of HEK 293 cells were probed with B3GALNT2. The cellular extracts were probed with B3GALNT2 1) in the presence of UDP-GalNAz, 2) in the presence of UDP-GalNAz after O-GlcNAcylation through in vitro OGT treatment in the presence of UDP-GlcNAc, and 3) in the presence of UDP-GalNAz after the removal of O-GlcNAc through OGA treatment. To demonstrate that OGT can be used to detect open O-GlcNAc sites on unknown proteins, nuclear and cytoplasmic extracts of HEK 293 cells were also probed with OGT in the presence of UDP-GlcNAz, as well as in the presence of OGA and UDP-GlcNAz.

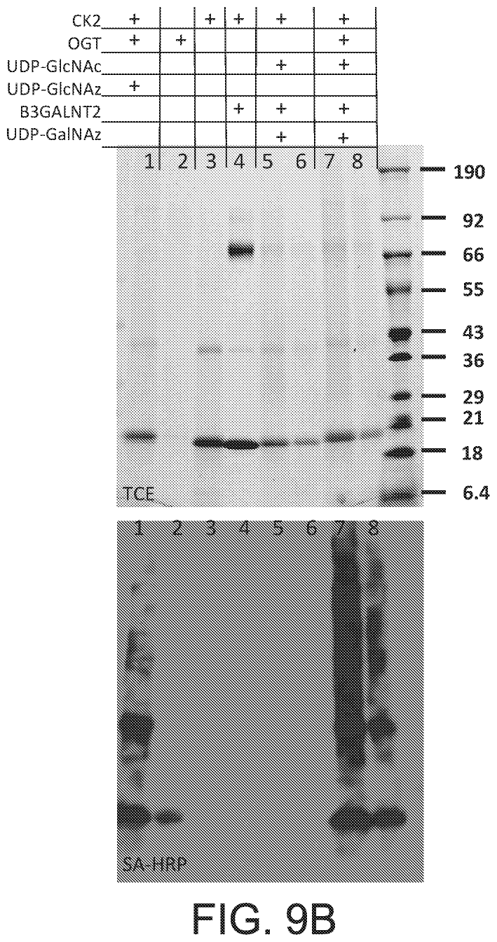

After the incorporation of GlcNAz or GalNAz into open or closed O-GlcNAc sites, respectively, a biotin moiety (biotin alkyne adduct) was conjugated to the GlcNAz or GalNAz via a click chemistry reaction to form labeled closed O-GlcNAc sites and labeled open O-GlcNAc sites, respectively. The samples were separated on 12% SDS-PAGE gels and visualized with trichloral ethanol (TCE) under UV, as shown in the upper panels of FIGS. 10A-10B. The gels were then blotted to nitrocellulose membranes and detected with strep-HRP, as shown in the lower panels of FIGS. 10A-10B.

FIG. 10A shows Western blot detection of closed O-GlcNAc sites using B3GALNT2. Nuclear extract was used in lanes 4-6 and cytoplasmic extract was used in lanes 7-9. The extracts in lanes 4 and 7 were probed with B3GALNT2 in the presence of UDP-GalNAz. The extracts in lanes 5 and 8 were probed with B3GALNT2 in the presence of UDP-GalNAz after O-GlcNAcylation through in vitro OGT treatment in the presence of UDP-GlcNAc. The extracts in lanes 6 and 9 were probed with B3GALNT2 in the presence of UDP-GalNAz after the removal of O-GlcNAc through OGA treatment. FIG. 10B shows Western blot detection of open O-GlcNAc sites using OGT. Nuclear extract was used in lanes a-c and cytoplasmic extract was used in lanes d-f. The extracts in lanes a, b, d, and e were probed with OGT in the presence of UDP-GlcNAz, with double the amount of OGT added in lanes b and e. The extracts in lanes c and f were probed with OGT in the presence of UDP-GlcNAz after treatment with OGA.

A comparison of lanes 5 and 8 to lanes 4 and 7 in FIG. 10A shows that OGT treatment increases the signals detected by B3GALNT2 significantly on both cytoplasmic and nuclear extracts, and particularly on nuclear extracts. This shows that there are more open sites than closed sites for O-GlcNAc on both extracts. On the contrary, as shown in lanes 6 and 9 of FIG. 10A and lanes c and fin FIG. B, pretreatment with OGA significantly reduced the signals detected by B3GALNT2 on both nuclear and cytoplasmic extracts. This confirms that B3GALNT2 detects closed O-GlcNAc sites. When open sites for O-GlcNAc on nuclear and cytoplasmic extracts of HEK 293 cells were probed with OGT, the signals observed were much stronger than those detected directly by B3GALNT2 (compare lanes 4 and 7 in FIG. 10A to lanes a and d of FIG. 10B, respectively). This also confirms that there are more open sites than closed sites for O-GlcNAc on both extracts.

### Example 5: Detection of O-GlcNAc Sites in CHO-K1 Cells to Determine Modification Degree

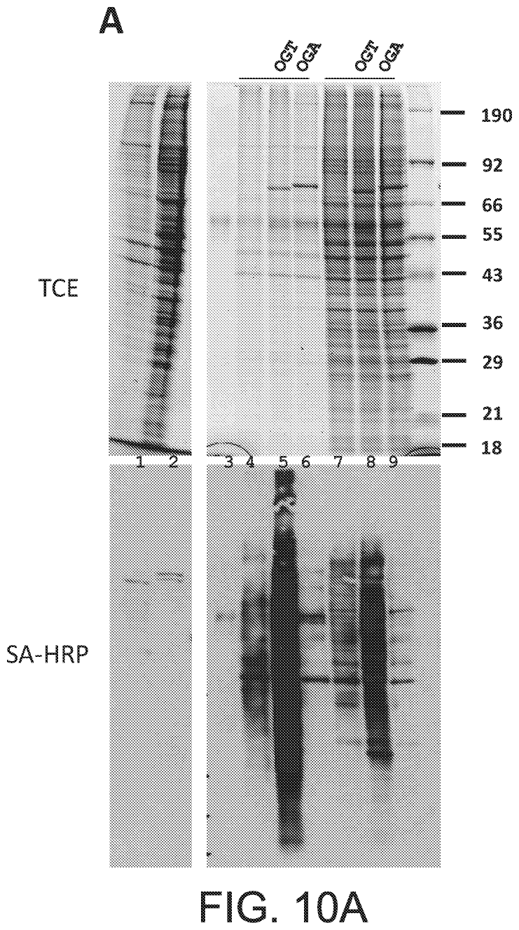

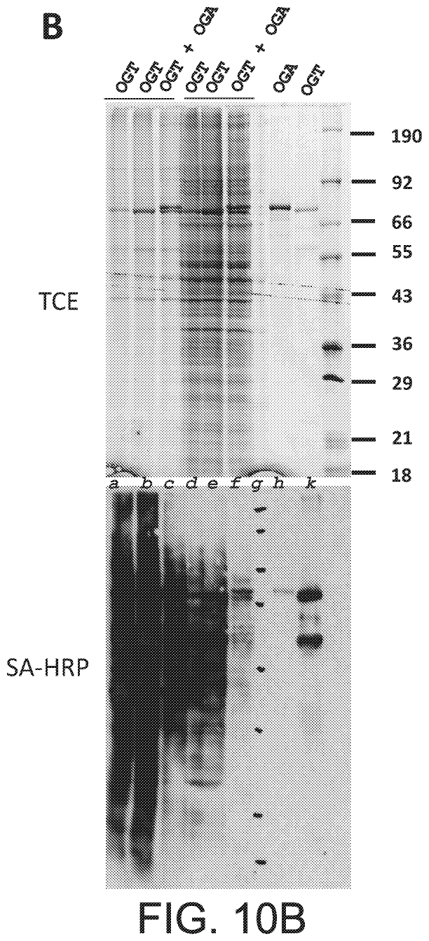

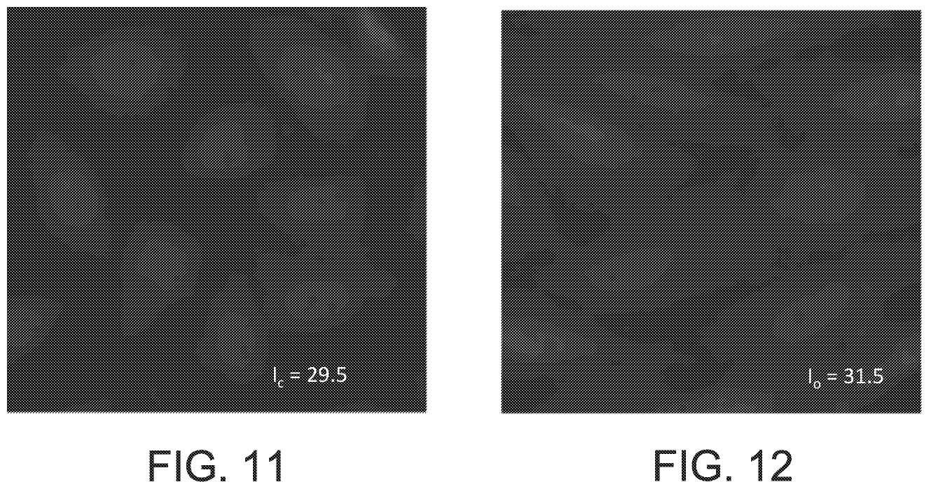

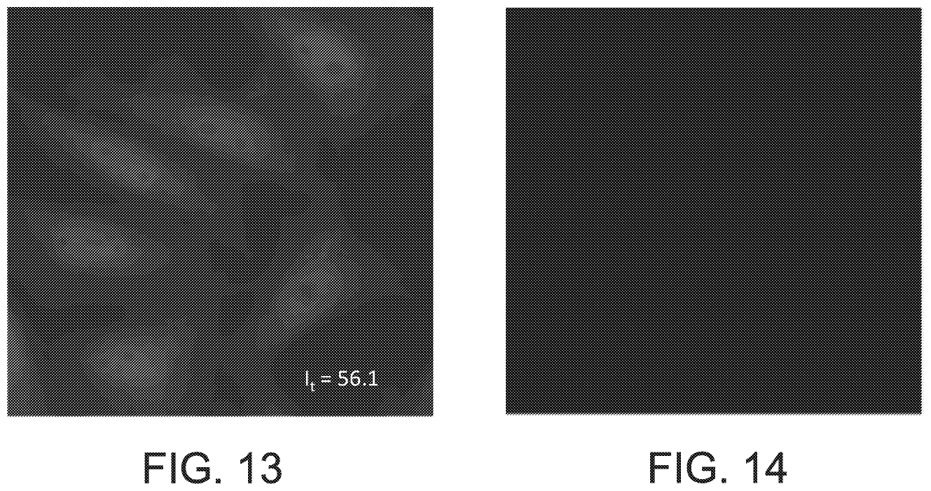

While detection of O-GlcNAc on proteins allows for a better understanding of its biological roles in individual proteins, determining modification degree by detecting and imaging O-GlcNAc sites inside of cells can provide a better understanding of how O-GlcNAc levels change in response to external stimulation or due to cell growth or cell migration. Both closed and open sites for O-GlcNAc were detected and imaged in CHO-K1 cells using B3GALNT2 and OGT respectively. CHO-K1 cells were grown on a 96-well plate to confluence and then fixed with 4% paraformaldehyde. After the introduction of clickable sugars (GlcNAz or GalNAz) with B3GALNT2 or OGT, the cells were tagged with biotin through a click chemistry reaction. Streptavidin-Alexa Fluor® 555 was subsequently bound to the conjugated biotin molecules, which resulted in fluorescently labeled open or closed O-GlcNAc sites. The fluorescently labeled cells were then imaged as described above. FIG. 11 is an image of CHO-K1 cells imaged for closed O-GlcNAc sites with B3GALNT2. FIG. 12 is an image of CHO-K1 cells imaged for open O-GlcNAc sites with OGT. FIG. 13 is an image of CHO-K1 cells imaged for total O-GlcNAc sites with B3GALNT2 after treatment with OGT in the presence of UDP-GlcNAc. FIG. 14 is an image of CHO-K1 cells imaged for closed O-GlcNAc sites with B3GALNT2 after treatment with OGA.

Imaging with B3GALNT2 revealed that closed O-GlcNAc sites are concentrated in nuclei (FIG. 11), which is consistent to previous reports for O-GlcNAc staining on mesenchymal stem cells and HUVEC cell staining. In contrast, imaging with OGT revealed that open O-GlcNAc sites were more evenly spread across the nuclei and cytoplasm (FIG. 12). When CHO-K1 cells were first treated with OGT extensively in the presence of UDP-GlcNAc to convert open sites to closed sites, and then imaged with B3GALNT2, significant enhancement of signal intensity was observed (FIG. 13). When O-GlcNAc was first removed with OGA and then imaged with B3GALNT2, almost entire signal was abolished (FIG. 14), confirming again that B3GALNT2 detection is specific to closed O-GlcNAc sites.

To determine modification degree of O-GlcNAc in the nuclei of the CHO-K1 cells, a region of interest (ROI) was selected on the nuclei of each visible cells in the panels in FIGS. 11, 12 and 13 and the average intensity (I) was measured using ImageJ. As expected, the sum of the intensity of closed O-GlcNAc sites in FIG. 11 (Ic) and the intensity of open O-GlcNAc sites in FIG. 12 (Io) (Ic+Io=61) is very close to that of the intensity of the total O-GlcNAc sites in FIG. 13 (It=56.1).

Assuming the signal intensity of the cells are directly proportional to the number of O-GlcNAc sites detected, based on the definition of modification degree discussed with respect to FIGS. 1-5 above, the modification degree (d) of O-GlcNAc in the nuclei of CHO-K1 cells was determined to be 52.6% (d=Ic/It=29.5/56.1=52.6%).

1. A method of detecting closed O-GlcNAc sites in a biological sample, the method comprising:
providing a biological sample;
treating the biological sample with β-1,3-N-acetylgalactosaminyltransferase (B3GALNT2) to incorporate GalNAz into closed O-GlcNAc sites in the sample, wherein the GalNAz comprises a click chemistry moiety;
adding a label to the biological sample, wherein the label comprises a click chemistry moiety that reacts to the click chemistry moiety of the GalNAz such that the label attaches to the GalNAz to form labeled closed O-GlcNAc sites; and
detecting the labeled closed O-GlcNAc sites.

2. The method of claim 1, wherein the biological sample comprises a purified protein, a cell, a cellular extract, or tissue.

3. The method of claim 1, wherein the B3GALNT2 comprises recombinant B3GALNT2.

4. The method of claim 1, wherein the label comprises a fluorescent label, a colorimetric label, a biotin label, or an enzyme label.

5. The method of claim 4, wherein the labeled closed O-GlcNAc sites are detected using a Western blot, a fluorescent imaging device, a colorimetric imaging device, or ELISA.

6. An in vitro method of detecting open O-GlcNAc sites in a biological sample, the method comprising:
providing a biological sample;
treating the sample with O-GlcNAc transferase (OGT) to incorporate GlcNAz into open O-GlcNAc sites in the biological sample, wherein the GlcNAz comprises a click chemistry moiety;
adding a label to the biological sample, wherein the label comprises a click chemistry moiety that reacts to the click chemistry moiety of the GlcNAz such that the label attaches to the GlcNAz to form labeled open O-GlcNAc sites; and
detecting the labeled open O-GlcNAc sites.

7. The method of claim 6, wherein the biological sample comprises a purified protein, a cell, a cellular extract, or tissue.

8. The method of claim 6, wherein the OGT comprises recombinant OGT.

9. The method of claim 6, wherein the label comprises a fluorescent label, a colorimetric label, a biotin label, or an enzyme label.

10. The method of claim 9, wherein the labeled open O-GlcNAc sites are detected using a Western blot, a fluorescent imaging device, a colorimetric imaging device, or ELISA.

11. The method of claim 1, the method further comprising:
treating the biological sample with OGT to convert open O-GlcNAc sites to closed O-GlcNAc sites.

12.-15. (canceled)

16. The method of claim 1, the method further comprising:
obtaining a second sample from the biological sample prior to treating the sample with B3GALNT2;
treating the second sample with O-GlcNAc transferase (OGT) to convert open O-GlcNAc sites to closed O-GlcNAc sites;
treating the second sample with B3GALNT2 to incorporate a GalNAz into the closed O-GlcNAc sites in the second sample, wherein the GalNAz comprises a click chemistry moiety;
adding a label to the second sample, wherein the label comprises a click chemistry moiety that reacts to the click chemistry moiety of the GalNAz such that the label attaches to the GalNAz to form labeled closed O-GlcNAc sites;
detecting the labeled closed O-GlcNAc sites in the second sample, wherein the labeled closed O-GlcNAc sites in the second sample correspond to the number of total O-GlcNAc sites in the biological sample; and
comparing the number of labeled closed O-GlcNAc sites in the biological sample to the number of total O-GlcNAc sites in the biological sample to determine a percentage of closed O-GlcNAc sites in the biological sample, wherein the percentage corresponds to the modification degree of O-GlcNAc in the biological sample.

17.-19. (canceled)

20. The method of claim 16, wherein the labeled closed O-GlcNAc sites in the biological sample and in the second sample are detected using a Western blot, a fluorescent imaging device, a colorimetric imaging device, or ELISA.

21. The method of claim 16, wherein the biological sample is from a patient, and wherein the method further comprises diagnosing the patient with diabetes if the modification degree of O-GlcNAc in the biological sample meets a threshold modification degree of O-GlcNAc.

22. The method of claim 21, the method further comprising treating the patient for diabetes.

23. The method of claim 16, wherein the biological sample is from a patient, and wherein the method further comprises diagnosing the-patient with cancer if the modification degree of O-GlcNAc in the biological sample meets a threshold modification degree of O-GlcNAc.

24. The method of claim 23, the method further comprising treating the patient for cancer.

25. A kit for in vitro detection of O-GlcNAc sites in a biological sample, the kit comprising:
β-1,3-N-acetylgalactosaminyltransferase (B3GALNT2) or O-GlcNAc transferase (OGT) or both;
UDP-GalNAz with a click chemistry moiety, UDP-GlcNAz with a click chemistry moiety, or UDP-GlcNAc, or a combination thereof;
a label comprising
a click chemistry moiety that reacts to the click chemistry moiety of UDP-GalNAz with a click chemistry moiety, or
a click chemistry moiety that reacts to the click chemistry moiety of UDP-GlcNAz with a click chemistry moiety; and
click chemistry reagents.

26. The kit of claim 25, wherein B3GALNT2 is recombinant or OGT is recombinant or both B3GALNT2 and OGT are recombinant.

27. The kit of claim 25, wherein the label is a fluorescent label, a colorimetric label, or a biotin label.

28.-33. (canceled)
